(19) United States
(12) Patent Application Publication
(10) Pub. No.: US 2018/0369166 A1
(43) Pub. Date: Dec. 27, 2018

(54) GARCINOL COMPOSITIONS FOR THERAPEUTIC MANAGEMENT OF ENDOPLASMIC RETICULUM STRESS

(71) Applicants: Muhammed Majeed, Edison, NJ (US); Kalyanam Nagabhushanam, East Windsor, NJ (US); Lakshmi Mundkur, Bangalore, IN (US)

(72) Inventors: Muhammed Majeed, Edison, NJ (US); Kalyanam Nagabhushanam, East Windsor, NJ (US); Lakshmi Mundkur, Bangalore, IN (US)

(21) Appl. No.: 16/015,503

(22) Filed: Jun. 22, 2018

Related U.S. Application Data
(60) Provisional application No. 62/523,592, filed on Jun. 22, 2017.

(51) Int. Cl. A61K 31/122; A61P 1/18; A61P 1/16

(52) U.S. Cl. CPC A61K 31/122; A61P 1/16; A61P 1/18

(57) ABSTRACT

Disclosed is the use of garcinol for the therapeutic management of ER stress. More specifically, the invention discloses the ability of garcinol in decreasing ER stress and mitigating toxicity by reducing protein aggregation and decreasing expression of ER stress markers SXBP, GRP78 and ATF4, which indicates translational attenuation and recovery in hyperglycemia, paracetamol, alcohol, thapsigargin and high fat diet induced toxicity models.

### CROSS-REFERENCE TO RELATED APPLICATION

This is a non-provisional application claiming priority from U.S. provisional application No. 62/523,592 filed on 22 Jun. 2017.

### BACKGROUND OF THE INVENTION

### Field of Invention

The present invention relates to therapeutic interventions for endoplasmic reticulum stress (ER stress). More specifically, the present invention relates to the potential of garcinol as a therapeutic agent for acute ER stress as a transcription attenuator and transcription recovery agent.

### Description of Prior Art

Endoplasmic reticulum (ER) plays a critical role in cellular stress responses by the synthesis and processing of secretory and membrane proteins (Umut O″zcan et al., (2004), Endoplasmic Reticulum Stress Links Obesity, Insulin Action, and Type 2 Diabetes, Science; 306(5695):457-61). Molecular chaperones present in the ER, facilitate proper protein folding, maintaining them in a folded state, and preventing protein aggregate formation (Lee A. S., (2005), The ER chaperone and signaling regulator GRP78/BiP as a monitor of endoplasmic reticulum stress. Methods; 35(4)1373-381). To ensure proper protein folding, the ER lumen maintains a unique environment to establish a balance between the ER protein load and the capacity to handle this load. Physiological or pathological processes that disturb ER homeostasis cause ER stress and activate a set of signaling pathways known as the Unfolded Protein Response (UPR) to cope up with the stress.

The initial objective of the UPR is to re-establish homeostasis and alleviate ER stress through two mechanisms: (a) increasing folding capacity via expression of protein-folding chaperones and (b) downregulation of ER protein by inhibiting general protein translation and promoting the degradation of misfolded proteins. Under prolonged or severe stress, the UPR initiates apoptosis and cell death. UPR-mediated cell death may contribute to the pathogenesis of many diseases including cancer, type 2 diabetes, neurodegeneration, and atherosclerosis (Tabas and Ron (2011), Integrating the mechanisms of apoptosis induced by endoplasmic reticulum stress. Nat Cell Biol. 13(3):184-90; Marciniak and Ron, (2006) Endoplasmic reticulum stress signaling in disease., Physiol Rev.; 86(4):1133-49). This complex cellular response is mediated initially by three molecules, PKR like ER kinase (PERK), activated transcription factor 6 (ATF6), and Inositol-requiring enzyme 1 (IRE1).

Under normal conditions, the PERK, IRE1, and ATF6 are bound to ER chaperone4 GRP78 (glucose-regulated protein); However, upon accumulation of unfolded proteins, GRP78 dissociates from these molecules, leading to their activation. IRE1 has a cytoplasmic endoribonuclease domain, which, upon activation, splices and enables the translation of the mRNA encoding X-box binding protein-1 (XBP1). Spliced XBP1 (XBP1s) is a transcription factor that induces many essential UPR genes that increase ER folding capacity and expand ER membrane surface area. Activated PERK phosphorylates eukaryotic translation initiation factor 2 alpha (eIF2α), which results in global translational attenuation and reduced ER protein load. Phosphorylated eIF2α promotes the translation of ATF4 (activating transcription factor-4), which induces the UPR effector CHOP which triggers apoptosis through a number of mechanisms. ATF6 translocates to the Golgi complex, where it gets cleaved by site 1 and site 2 proteases. The resultant transcription factor then migrates to the nucleus to increase the expression of ER chaperones such as Grp78.

Certain pathological stress conditions are implicated in the disruption of ER homeostasis, leading to the accumulation of unfolded or misfolded proteins in the ER lumen (R. Y. Hampton, Curr. Biol. 10, R518 (2000), K. Mori, Cell 101, 451 (2000), H. P. Harding, M. Calfon, F. Urano, I. Novoa, D. Ron, Annu. Rev. Cell Dev. Biol. 18, 575 (2002). Among pathological conditions implicated for ER stress, glucose or nutrient deprivation, viral infections, lipids, increased synthesis of secretory proteins, and expression of mutant or misfolded proteins assume tremendous importance (Y. Ma, L. M. Hendershot, Cell 107, 827 (2001), R. J. Kaufman et al., Nature Rev. Mol. Cell Biol. 3, 411 (2002) and I. Kharroubi et al., Endocrinology (2004); published online 5 Aug. 2004 (10.1210/en.2004-0478)). The following prior arts describes in detail regarding the role of ER stress in different diseases.
1. Umut O″zcan et al., (2004), Endoplasmic Reticulum Stress Links Obesity, Insulin Action, and Type 2 Diabetes, Science; 306(5695):457-61) Diabetes 2008 September; 57(9):2438-44.
2. Yoshida H (2007) ER stress and diseases, FEBS J.; 274(3):630-58.
3. Asselah et. al., (2010) In vivo hepatic endoplasmic reticulum stress in patients with chronic hepatitis C. J Pathol. 221(3):264-74.
4. Ji et al., (2003), Betaine decreases hyperhomocysteinemia, endoplasmic reticulum stress, and liver injury in alcohol-fed mice. Gastroenterology. 124(5):1468-99.
5. Hoozemans et al., (2005). The unfolded protein response is activated in Alzheimer's disease. Acta Neuropathol.; 110(2):165-72.

Thus, therapeutic interventions that have the ability to regulate the ER stress response are expected to offer new opportunities for the management of the cluster of pathologies that are implicated in ER stress, most importantly the metabolic syndrome.

ER stress has 2 distinct perspectives (Oyadomari et al., “Roles of CHOP/GADD153 in encloplasmic reticulum stress”, Cell Death and Differentiation (2004) 11, 381-389)
1. An acute perspective that allows remodelling phase where translational attenuation reducing the load of new protein synthesis and preventing further accumulation of unfolded proteins occur. This mechanism provides a recovery phenomenon for the cells to survive (survival signal); and
2. A chronic perspective where ER stress leads to the activation of apoptotic signals leading to cell death.

Therapeutic interventions may be useful in either phases or in one of the phases. Evidence of such therapeutic interventions thus forms the corner stone for the manaaement of ER stress. Garcinol as an apoptotic signalling agent in ER stress has been reported by Cheng et al, “Garcinol inhibits cell growth in hepatocellular carcinoma Hep3B cells through induction of ROS-dependent apoptosis”, Food Funct. 2010 December; 1(3):301-7. Garcinol's role in apoptotic signalling through the activation of DNA damage-inducible gene 153 (GADD153) has been reported in this piece of technical literature.

However, the effect of garcinol as a cell survival signalling agent in terms of modulating translational attenuation in acute ER stress has never been reported before. Chronic ER stress has to be handled only by the induction of apoptosis, where recovery and survival of the cell has no chance. Therefore, therapeutically managing acute ER stress is the mainstay for ER stress management. Therefore, scientific evidence in this front forms a novel, non-obvious and industrially applicable addition to the existing knowledge networks on the therapeutic applications of garcinol.

It is thus the principle objective of the present invention to disclose the potential of garcinol as a modulator of acute ER stress through translational attenuation mechanism and a facilitator of translational recovery.

The present invention fulfils the aforesaid objective and provides further related advantages.

### SUMMARY OF THE INVENTION

The present invention discloses the ability of garcinol for the therapeutic management of ER stress. More specifically, the invention discloses the ability of garcinol in decreasing ER stress and mitigating toxicity by reducing protein aggregation and decreasing expression of ER stress markers SXBP, GRP78 and ATF4, which indicates translational attenuation and recovery in hyperglycemia, paracetamol, alcohol, thapsigargin and high fat diet induced toxicity models.

Other features and advantages of the present invention will become apparent from the following more detailed description, taken in conjunction with the accompanying drawings, which illustrate, by way of example, the principle of the invention.

### BRIEF DESCRIPTION OF DRAWINGS

FIG. 1 is the graphical representation of protection against glucose induced toxicity in pancreatic beta cell line by garcinol.

FIG. 2 is a flow cytometric image showing the reduction in protein aggregation and reduction in ER stress garcinol in Minh pancreatic beta cells (glucose induced toxicity).

FIG. 3 is a graphical representation showing dose dependant reduction in glucose in serum of mice supplemented with garcinol (high fat induced diabetic condition in mice).

FIG. 4 is a graphical representation showing decrease in the expression of ER stress markers in pancreas of animals supplemented with garcinol (high fat induced diabetic condition in mice).

FIG. 5a is a graphical representation showing percentage hepatoprotection by garcinol in paracetamol induced liver toxicity (Prophylactic) in study animals.

FIG. 5b is a graphical representation showing percentage hepatoprotection by garcinol in paracetamol induced liver toxicity (Therapeutic) in study animals.

FIG. 6 is a graphical representation showing percentage hepatoprotection by garcinol in alcohol induced liver toxicity in HepG2 Cells

FIG. 7a is a graphical representation showing percentage reduction in protein aggregation by garcinol estimated by means thioflavin staining in thapsigargin induced human liver cells. US—unstressed cells, TG—Thapsigargin, G5, G10—Garcinol 5 and 10 μg/ml respectively, PBA: phenyl butyric acid (positive control)

FIG. 7b is the flow cytometric image showing percentage reduction in protein aggregation by garcinol estimated by means thioflavin staining in thapsigargin induced human liver cells.

FIG. 7c is a graphical representation showing percentage reduction in protein aggregation by garcinol estimated by means thioflavin staining in thapsigargin induced primary mouse hepatocytes.

FIG. 8 is a graphical representation showing decrease in the expression of ER stress markers by garcinol hepatocytes treated with thapsigargin. TG-Thapsigargin, G5, G10—Garcinol 5 and 10 ug/ml respectively, PBA: phenyl butyric acid (positive control).

FIG. 9 is a graphical representation showing dose dependant decrease in liver weight of animals by garcinol in animals administered with high fat diet.

FIG. 10 is a graphical representation showing decrease in the expression of ER stress markers in pancrease of high fat diet administered animals supplemented with garcinol.

FIG. 11a is a graphical representation showing decrease in the expression of ER stress markers in adipocytes treated with thapsigargin. TG—Thapsigargin, G5, G10—Garcinol 5 and 10 ug/ml respectively, PBA: phenyl butyric acid (positive control).

FIG. 11b is a graphical representation showing decrease in the expression of ER stress markers in fat pads of high fat diet administered animals supplemented with garcinol.

### DESCRIPTION OF THE MOST PREFERRED EMBODIMENT

In the most preferred embodiment, the present invention relates to a method of treating endoplasmic reticulum stress in mammalian cells characterised by accumulation of unfolded or misfolded cellular protein transcripts, said method comprising step of treating said mammalian cells with effective concentrations of garcinol to bring about effects of attenuating the accumulation of said unfolded or misfolded protein transcripts. In a related embodiment, the effective concentration of garcinol is 2-10 μg/ml. In a related embodiment, the mammalian cells are preferably human cells.

In another preferred embodiment, the inventions disclose a method for reducing endoplasmic reticulum stress and related toxicity in mammals, said method comprising step of administering effective concentration of garcinol to said mammals to bring about a reduction in the toxicity and endoplasmic reticulum stress markers. In a related embodiment, ER stress is present in clinical conditions selected from the group consisting of, but not limited to, metabolic syndrome, diabetes, artherosclerosis, neurodegenerative disorders likes Alzheimer's disease and Parkinson's disease, alcoholic and non-alcoholic hepatic steatosis, cancer, viral infections, hyperglycemia, drug induced toxicity and obesity. In another related embodiment the markers of ER stress are selected from the group consisting of spliced x-box DNA binding protein (SXBP), activating transcription factor-4 (ATF-4) and glucose-regulated protein 78 (GRP78). In a related embodiment, the mammal is human. In another related embodiment, the effective concentration of garcinol is 0.1-5 mg/kg body weight. In another preferred embodiment, garcinol is formulated in a composition along with pharmaceutically/nutraceutically acceptable excipients, adjuvants, diluents or carriers and administered orally in the form of tablets, capsules, syrups, gummies, powders, suspensions, emulsions, chewables, candies and eatables.

In yet another most preferred embodiment, the present invention relates to a method of therapeutic management of encloplasmic reticulum stress induced metabolic syndrome, said method comprising step of administering effective concentrations of garcinol to said mammals to bring about effects of attenuating the accumulation of said unfolded or misfolded protein transcripts leading to symptoms of metabolic syndrome. In a related embodiment, metabolic syndrome is present in clinical conditions selected from the group consisting of diabetes, artherosclerosis, neurodegenerative disorders likes Alzheimer's disease and Parkinson's disease, alcoholic and non-alcoholic hepatic steatosis, cancer, viral infections, hyperglycemia, drug induced toxicity and obesity. In a related embodiment, the mammal is human. In another related embodiment, the effective concentration of garcinol is 0.1-5 mg/kg body weight. In another preferred embodiment, garcinol is formulated in a composition along with pharmaceutically/nutraceutically acceptable excipients, adjuvants, diluents or carriers and administered orally in the form of tablets, capsules, syrups, gummies, powders, suspensions, emulsions, chewables, candies and eatables.

The aforesaid most preferred embodiments incorporating the technical features and technical effects of instant invention, are explained through illustrative examples herein under.

### EXAMPLE 1

### Methods

Cell Lines

HepG2—human liver cell line, primary mouse hepatocytes. Min6—Mouse pancreatic beta cell line, 3T3 L1—mouse preadipocytes wee used for the experiments. Cells were maintained in DMEM containing 25 mM glucose with 10% heat-inactivated fetal calf serum with antibiotics at 37° C. and 5% CO2. When the cells were 70-80% confluent, they were trypsinized, washed and seeded in 96 well plates at a density of 5×103 cells per well. Stress was induced by Thapsigargin(1 uM) for 4 hours, glucose (50 and 100 mM) and alcohol (1000 mM) or paracetamol (4 mM) for 72 hours. Garcinol was added along with stressor as prophylactic treatment. As a therapeutic treatment, cells were exposed to stress agent 24 hours following which Garcinol was added to the cells. Protection was assessed by SRB assay after 72 hours of treatment

Animals—C57/BL6 mice, 6-8 weeks of age and 8 animals/Group were used for the study. Animals were housed under standard laboratory conditions, air-conditioned with adequate fresh air supply (12-15 Air changes per hour), room temperature 20.2-23.5° C. and relative humidity 58 64% with 12 hours fluorescent light and 12 hours dark cycle. The temperature and relative humidity was recorded once daily.

Feed: The animals were fed with Normal diet (9 kcal/day) and High fat diet (50 kcal/day) throughout the acclimatization and experimental period. Water was provided along with High Fat Diet to the animals throughout the acclimatization and experimental period. Water from water filter cum purifier was provided in animal feeding bottle with stainless steel sipper tubes. Garcinol was administered to the animals along with the high fat diet. The dosage calculation from animals to humans as described by Regan-Shaw et al,, (2007) Dose translation from animal to human studies revisited FASEB J. 22, 659-661 was used as reference.

All the studies were conducted according to the ethical guidelines of CPCSEA after obtaining necessary clearance from the committee (Approval No: 790/03/ac/CPCSEA).
a. In accordance with the recommendation of the Committee for the Purpose of Control and Supervision of Experiments on Animals (CPCSEA) guidelines for laboratory animal facility published in the gazette of India. Dec. 15, 1998.
b. The CPCSEA approval number for the present study (Anti-obesity activity) is SAC/IAEC/BC/2017/IP.-001.

At the end of the experimental period, the animals were sacrificed by cervical dislocation and organs collected to study the ER stress related gene expression and serum collected for biochemical analysis

RNA Extraction

Cells were harvested after second progression on day 7 and total RNA was extracted using the Trizol method. Extracted RNA was treated with DNAse I to remove any contaminating DNA and again extracted using phenol: chloroform: isoamyl alcohol extraction (24:25:1). Quality of RNA was determined by checking the absorbance at 260/280 nm using a Nanodrop (Thermo)

Gene Expression Studies in Mouse Fat Pad, Liver and Pancreas

The frozen organs from treated and untreated animals were collected in RNA later and frozen. Approximately 100 mg of the tissue was homogenized in ice and extracted with 1 ml Trizol as described earlier.

Quantitative Real Time PCR

2 μg of total RNA was taken for cDNA synthesis using SuperScript III First-Strand Synthesis System (Life Technologies). Quantitative RT-PCR analysis was performed to determine the expression of brown fat specific genes in Roche Light cycler 96 using SYBR Green master mix (Thermo Scientific). β actin was used as a house keeping gene The relative RNA abundance of genes associated with ER stress were normalized to the housekeeping β actin gene and expressed, as delta delta CT (equivalent to fold change transformed by Log2).

Methods for Measuring IRE1α Activation

IRE1α phosophorylation levels can be assessed by measuring spliced. XBP-1 product by RT PCR. PERK activation is measured by its phosphorylation levels or by measuring the ATF4 mRNA levels. Activation of ATF4 leads to increased transcription of a network of genes, including those encoding ER chaperones, such as BiP/GRP78. Thioflavin T (ThT) is a small molecule with fluorescence properties that has been shown to bind selectively to protein aggregates and can be analysed by Flow cytometry
Primer sequence: The primers used for the determining the expression of ER stress specific genes are given in table 1

TABLE 1
Primer sequence

| | |
|---|---|
| msXBP1 F | CTGAGTCCGAATCAGGTGCAG |
| msXBP1 R | GTCCATGGGAAGATGTTCTGG |
| mGRP78F | TTCAGCCAATTATCAGCAAACTCT |
| mGRP78F | TTTTCTGATGTATCCTCTTCACCAGT |
| mATF4 F | GGGTTCTGTCTTCCACTCCA |
| mATF4 R | AAGCAGCAGAGTCAGGCTTTC |

### EXAMPLE 2

### Results

Effect of Garcinol on Pancreatic ER Stress

Under high glucose concentration ER stress is induced in pancreatic beta cells in vitro. These cells are grown in 5-10 mM of Glucose as the glucose concentration increases, the cells are stressed and ultimately undergo apoptosis at higher concentration.

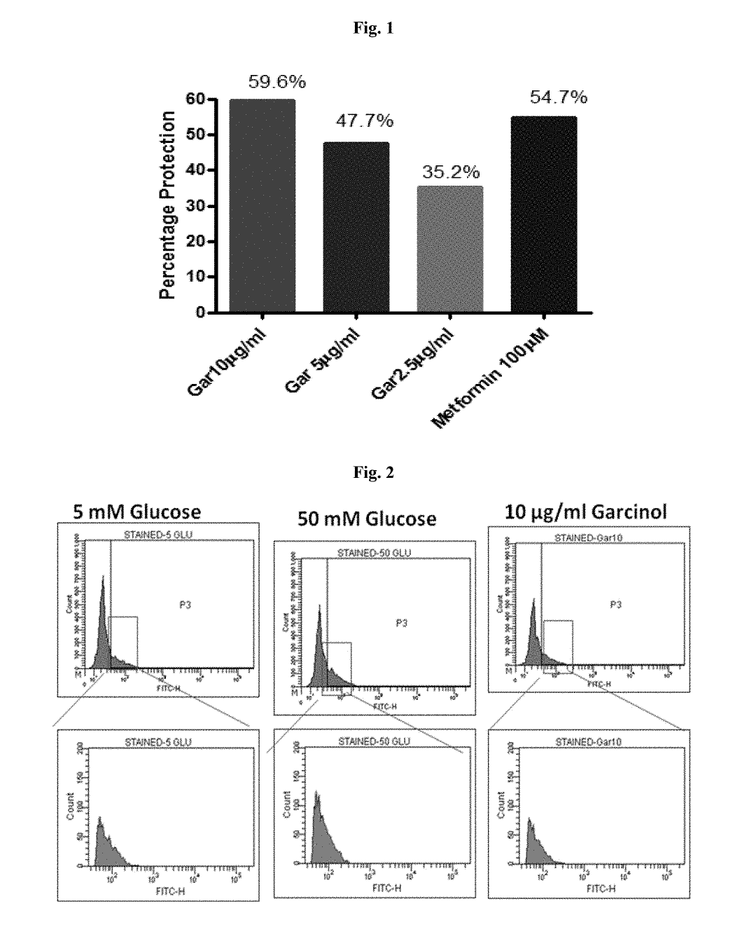

Garcinol treatment reduced the toxicity induced by Hyperglycemia in a dose dependent manner with an effect comparable to Metformin (100 μM) (FIG. 1), there by conferring Protection against Glucose induced Toxicity in Pancreatic Beta cell line. Garcinol treatment also reduced the protein aggregation induced by high concentration of Glucose in pancreatic cells (FIG. 2), and reducing the ER stress.

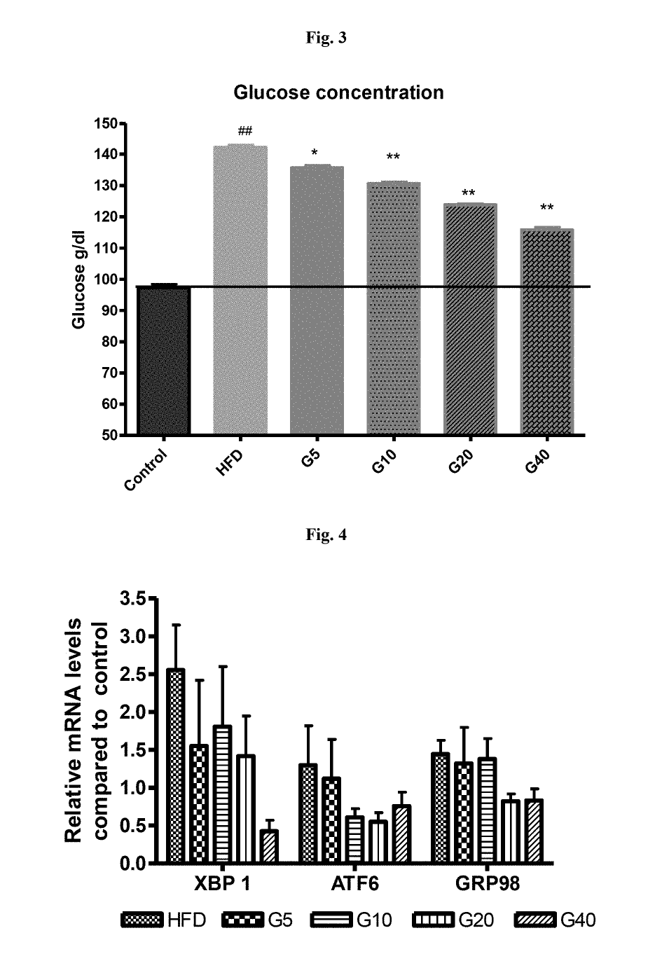

Garcinol also conferred protection against high fat induced pancreatic toxicity and serum glucose levels in mice. Garcinol reduced the serum glucose levels significantly (FIG. 3) suggesting it can be helpful in preventing obesity induced diabetes. The relative expression of ER stress markers were up regulated in pancreas of animals kept on high fat diet compared to control animals on chow diet feed. Garcinol treatment reduced the ER stress markers in HFD fed animals (FIG. 4). These results suggest that Garcinol treatment can effectively reduce ER stress induced by High fat diet in mouse pancreas which in turn results in lower serum glucose levels in these animals. Thus Garcinol can help in alleviating diabetes by reducing ER stress.

Effect of Garcinol on ER Stress in Liver

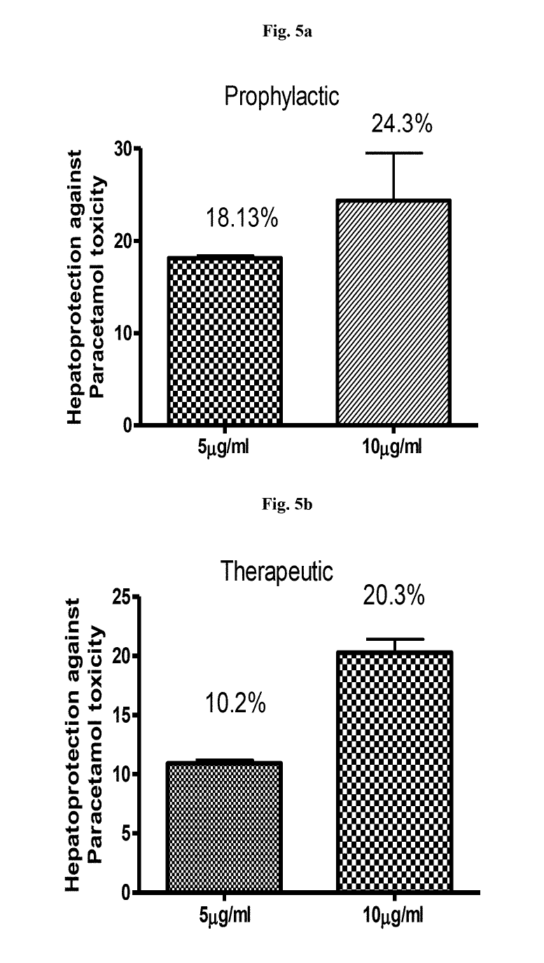

The effect of garcinol on paracetamol induced liver toxicity was measured. Garcinol protected liver cells against paracetamol induced toxicity as a prophylactic (FIG. 5a) and as a therapeutic (FIG. 5b) at concentrations of 5 μg/ml and 10 μg/ml.

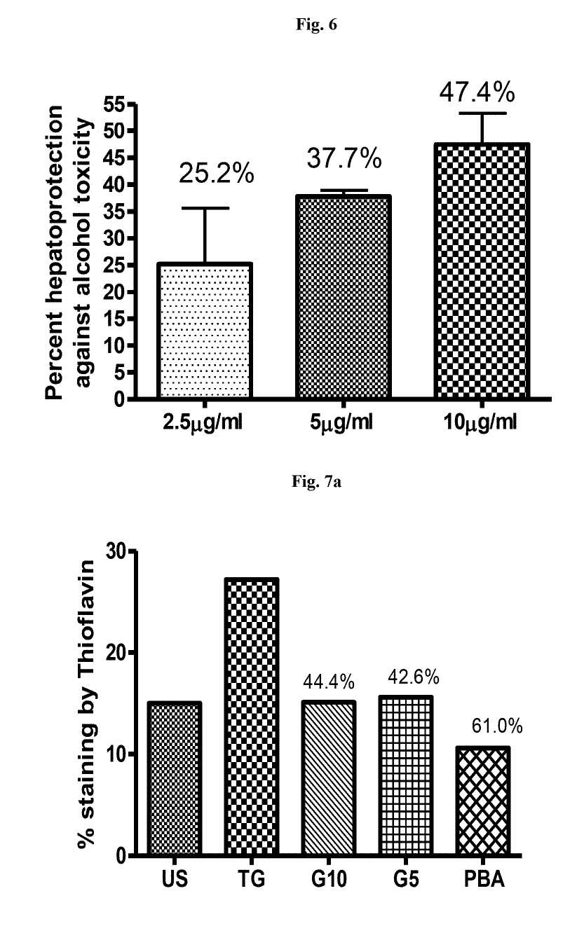

For evaluating alcohol induced toxicity, HepG2 Cells were exposed to 1000 mM of Ethanol for 72 hours in the presence of different concentrations of Garcinol and protection was assessed by SRB assay. Garcinol conferred hepatoprotection against alcohol induced toxicity by 47% at concentration of 10 μg/ml (FIG. 6).

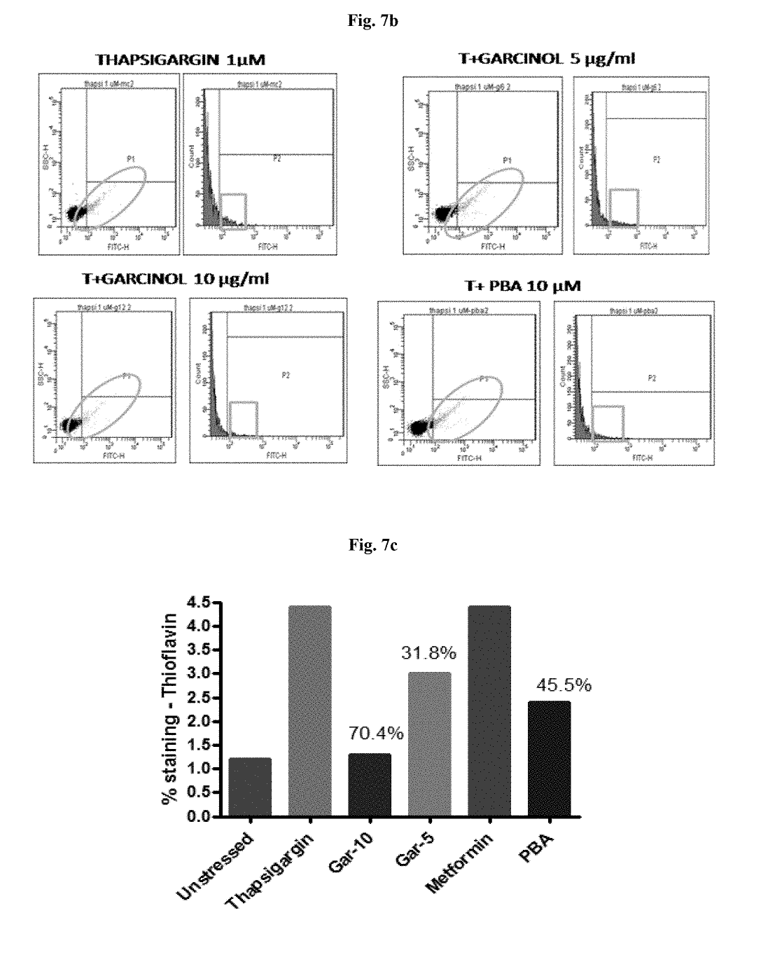

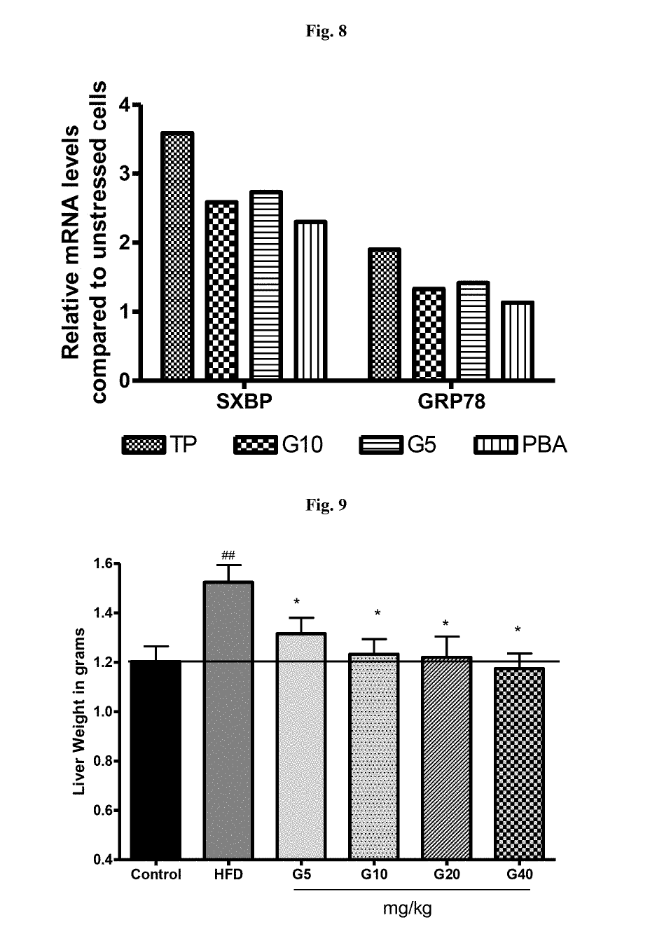

Effect of Garcinol on Thapsigargin induced ER stress in human liver cells was also evaluated. Human Liver cell line (HepG2) was maintained in DMEM containing 25 mM glucose with 10% heat-inactivated fetal calf serum with antibiotics at 37° C. and 5% CO2. When the cells were 70-80% confluent, they were trypsinized, washed and seeded in 96 well plates at a density of 5×10³ cells per well Primary Hepatocytes were isolated from mouse liver. Cells were cultured in DMEM for 3 hours with 1 μM Thapsigargin to induce ER stress. Garcinol, at 10 and 5 μg/ml was used along with Thapsigargin. Untreated cells were used as control and Phenyl butyric acid (PBA) was used as positive control. Treated and untreated cells were analysed by Flow cytometry and RT PCR. Garcinol significantly reduced, protein aggregation in human liver cells (FIGS. 7a and 7b) by 44.4% and in primary mouse hepatocytes (FIG. 7c) by 70.4% at a concentration of 10 μg/ml. Garcinol also reduced the expression of ER stress markers sXBP 1 and GRP78 (FIG. 8) indicating that garcinol is effective in reducing ER stress resulting due to increased protein aggregation.

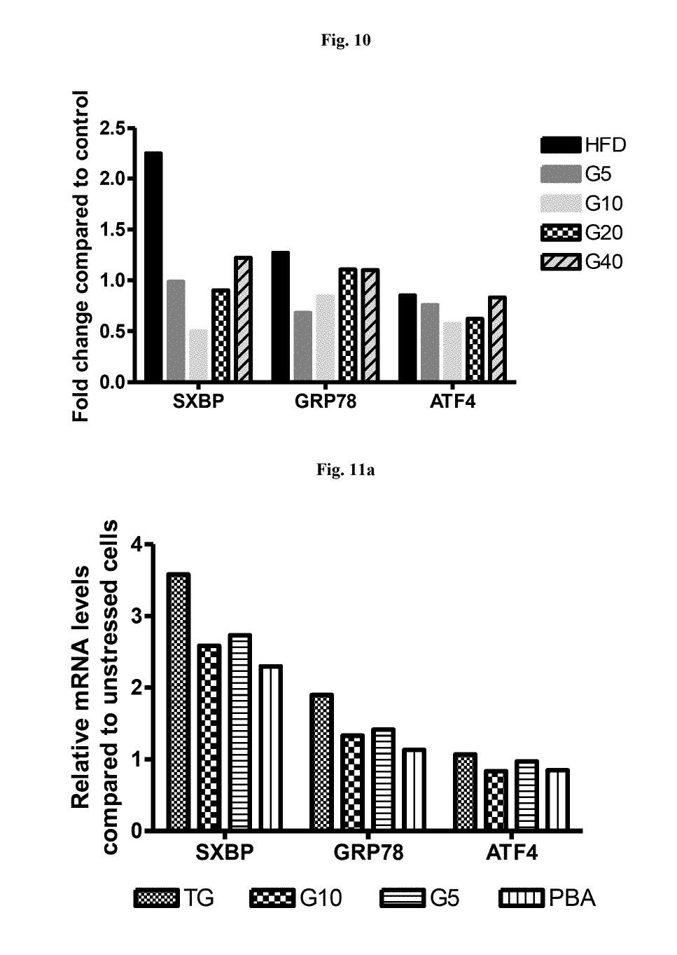

Garcinol reduced the liver weight significantly in high fat induced liver toxicity in mice (FIG. 9) suggesting it may be helpful in preventing obesity induced fatty liver. The expression of marker of ER stress—SXBP, GRP78 and ATF4 were also significantly reduced by garcinol in the high fat induced liver toxicity (FIG. 10) in mice model suggesting its use as a liver protection agent.

Effect of Garcinol on ER Stress in Adipocytes

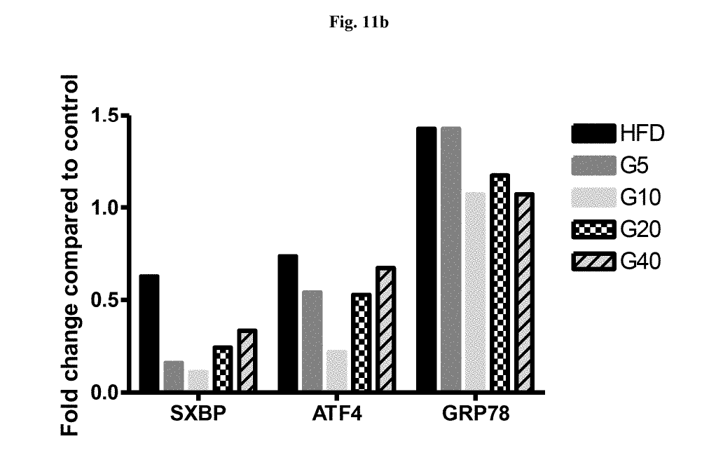

Garcinol significantly decreased the expression of stress markers—SXBP, GRP78 and ATF4 in both the adipocytes (FIG. 11a) and in the fat pads (FIG. 11b), indicating that Garcinol is effective in reducing the ER stress in High fat diet induced stress in adipose tissue of mice and reduces adipogenesis and obesity in mice.

These results suggest that Garcinol protects against type 2 diabetes, liver damage and adipogenesis by reducing ER stress in pancreas, liver and adipocytes. Owing to the ability of garcinol to mitigate ER stress in diabetes, liver toxicity and obesity (high fat diet), all of which are one of the important factors for the development of metabolic syndrome (Alberti et al., (2005), The metabolic syndrome—a new worldwide definition, The Lancet, Volume 366, No. 9491, p 1059-1062), garcinol reduces metabolic syndrome by the mechanism of reducing ER stress.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention.

We claim:

1. A method of treating endoplasmic reticulum stress in mammalian cells characterized by accumulation of unfolded or misfolded cellular protein transcripts, said method comprising step of treating said mammalian cells with effective concentrations of garcinol to bring about effects of attenuating the accumulation of said unfolded or misfolded protein transcripts.

2. The method as in claim 1, wherein the effective concentration of garcinol is 2-10 μg/ml.

3. The method as in claim 1, wherein the mammalian cells are preferably human cells.

4. A method for reducing the endoplasmic reticulum stress and related toxicity in mammals, said method comprising step of administering effective concentration of garcinol to said mammals to bring about a reduction in the toxicity and endoplasmic reticulum stress markers.

5. The method as in claim 4, wherein endoplasmic reticulum stress is present in clinical conditions selected from the group consisting of metabolic syndrome, diabetes, artherosclerosis, neurodegenerative disorders likes Alzheimer's disease and Parkinson's disease, alcoholic and non-alcoholic hepatic steatosis, cancer, viral infections, hyperglycemia, drug induced toxicity and obesity.

6. The method as in claim 4, wherein the markers of endoplasmic reticulum stress are selected from the group consisting of SXBP, ATF-4 and GRP78.

7. The method as in claim 4, wherein the effective concentration of garcinol is 1-5 mg/kg body weight.

8. The method as in claim 4, wherein the mammal is human.

9. The method as in claim 4, wherein garcinol is formulated in a composition along with pharmaceutically/nutraceutically acceptable excipients, adjuvants, diluents or carriers and administered orally in the form of tablets, capsules, syrups, gummies, powders, suspensions, emulsions, chewables, candies and eatables.

10. A method of therapeutic management of endoplasmic reticulum stress induced metabolic syndrome, said method comprising step of administering effective concentrations of garcinol to said mammals to bring about effects of attenuating the accumulation of said unfolded or misfolded protein transcripts leading to symptoms of metabolic syndrome.

11. The method as in claim 10, wherein metabolic syndrome is present in clinical conditions selected from the group consisting of diabetes, artherosclerosis, neurodegenerative disorders likes Alzheimer's disease and Parkinson's disease, alcoholic and non-alcoholic hepatic steatosis, cancer, viral infections, hyperglycemia, drug induced toxicity and obesity.

12. The method as in claim 10, wherein the effective concentration of garcinol is 0.1-5 mg/kg body weight.

13. The method as in claim 10, wherein the mammal is human.

14. The method as in claim 10, wherein garcinol is formulated in a composition alone with pharmaceutically/nutraceutically acceptable excipients, adjuvants, diluents or carriers and administered orally in the form of tablets, capsules, syrups, gummies, powders, suspensions, emulsions, chewables, candies and eatables.
